(19) United States
(12) Patent Application Publication
(10) Pub. No.: US 2024/0041904 A1
(43) Pub. Date: Feb. 8, 2024

(54) COMPOSITIONS COMPRISING CANNABINOID ACID ESTERS

(71) Applicant: BUZZELET DEVELOPMENT AND TECHNOLOGIES LTD., Or Akiva (IL)

(72) Inventor: Aharon EYAL, Jerusalem (IL)

(21) Appl. No.: 18/266,618

(22) PCT Filed: Dec. 22, 2021
(86) PCT No.: PCT/IB2021/062154

§ 371 Date: Jun. 12, 2023

Related U.S. Application Data
(60) Provisional application No. 63/130,448, filed on Dec. 24, 2020.

(51) Int. Cl. A61K 31/00; A61K 31/047

(52) U.S. Cl. CPC A61K 31/658; A61K 31/047

(57) ABSTRACT

Provided is a composition comprising an ester derived from at least one acid form of a cannabinoid (such as one selected from the group consisting of cannabigerolic acid (CBGA), tetrahydrocannabinolic acid (THCA), cannabidiolic acid (CBDA), cannabichromenic acid (CBCA), cannabigerovarinic acid (CBGVA), tetrahydrocanabivarinic acid (CBCVA), cannabidivarinic acid (CBDVA) and cannabichromevarinic acid (CBCVA) and combinations thereof) and a water-soluble alkanol and uses thereof in therapy.

### FIELD OF THE INVENTION

The field of art to which this invention generally pertains is cannabinoid compositions, and more specifically to compositions comprising an ester of at least one acid form of a cannabinoid and a water-soluble alkanol.

### BACKGROUND OF THE INVENTION

Cannabis is a complex plant comprising over 400 chemical entities, some of which have opposing pharmacological effects. The main subspecies are Cannabis indica and Cannabis sativa, of which Cannabis sativa is the more commonly known.

The main active components of cannabis are named cannabinoids. So far, more than 100 cannabinoids have been discovered. Individual cannabis plants may contain multiple cannabinoids, sometimes dozens of them. The best known cannabinoids are tetrahydrocannabinol (THC) and cannabidiol (CBD). Others include cannabigerol (CBG), cannabichromene (CBC) tetrahydrocannabivarin (THCV) Cannabidivarin (CBDV) and cannabinol (CBN). Cannabinoids are found in various parts of the cannabis plant. Typically, their concentration is greatest in the flower (the bud), particularly in the trichomes.

In the naturally occurring cannabis plant, these cannabinoids are mostly synthesized and accumulated in their acid form as cannabinoid acids such as CBDa and THCa. For most therapeutic applications, cannabinoids are heat treated to effect thermal decarboxylation of the acid form of the cannabinoids into the decarboxylated form, such as CBD or THC.

### SUMMARY OF THE INVENTION

According to an aspect of some embodiments of the present invention, there is provided a composition comprising an ester derived from at least one acid form of a cannabinoid and a water-soluble alkanol.

According to an aspect of some embodiments of the present invention, there is provided the composition as disclosed herein for use in therapy.

According to an aspect of some embodiments of the present invention, there is provided a method of treating a subject in need thereof, comprising administering the composition as disclosed herein.

### DETAILED DESCRIPTION OF THE INVENTION

The present invention, in at least some embodiments, relates to composition comprising an ester derived from at least one acid form of a cannabinoid and a water-soluble alkanol, and to uses thereof in therapy.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used herein, the term “cannabinoid” refers to a compound that affects the endocannabinoid system. Cannabinoids are agonists or antagonists to receptors in the endocannabinoid system.

As used herein, the term “THC” refers to THCa (tetrahydrocannabinolic acid) and/or to THC (tetrahydrocannabinol) unless indicated otherwise. As used herein, the term “CBD” refers to CBDa (cannabidiolic acid) and/or to CBD (cannabidiol) unless indicated otherwise. As used herein, the term “CBG” refers to CBGa (cannabigerolic acid) and/or to CBG (cannabigerol) unless indicated otherwise. As used herein, the term “CBN” refers to CBNa (Cannabinolic acid) and/or to CBN (cannabinol) unless indicated otherwise. As used herein, the term “CBC” refers to CBCa (cannabichromenic acid) and/or to CBC (Cannabichromene) unless indicated otherwise. As used herein, the term “CBL” refers to CBLa (Cannabicycol acid) and/or to CBL (Cannabicyclol) unless indicated otherwise. As used herein, the term “THCV” refers to THCVa (tetrahydrocannabivarin acid) and/or to THCV (tetrahydrocannabivarin) unless indicated otherwise. As used herein, the term “CBDV” refers to CBDVa (cannabigerovarin acid).

As used herein, the term “administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, intramuscular, intraosseous, intrathecal, and transdermal, or combinations thereof. “Administering” can also include providing a different compound that when ingested or delivered as above will necessarily transform into the compound that is desired to be administered, this type of “different compound” is often being referred to as a “Prodrug”. “Administering” can also include prescribing or filling a prescription for a dosage form comprising a particular compound. “Administering” can also include providing directions to carry out a method involving a particular compound or a dosage form comprising the compound or compounds.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value relating to a concentration of a terpene is preceded by the term “about”, the term “about” is intended to indicate +/−30% of that value.

As used herein, when a numerical value relating to a concentration of a cannabinoid is preceded by the term “about”, the term “about” is intended to indicate +/−20% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

The present inventors have found that a composition comprising an ester of an acid form of a cannabinoid is surprisingly useful in therapy, providing a highly stable compound which is hydrolyzed to an active form in the body of a subject subsequent to administration.

Furthermore, such compositions have greater water solubility than the acid form itself so are more useful for the preparation of compositions in aqueous media.

According to an aspect of some embodiments of the present invention, there is provided a composition comprising an ester derived from at least one (such as one, two, three or more) acid form(s) of a cannabinoid and a water-soluble alkanol.

According to some embodiments, said at least one acid form of a cannabinoid is selected from the group consisting of cannabigerolic acid (CBGA), tetrahydrocannabinolic acid (THCA), cannabidiolic acid (CBDA), cannabichromenic acid (CBCA), cannabigerovarinic acid (CBGVA), tetrahydrocanabivarinic acid (CBCVA), cannabidivarinic acid (CBDVA) and cannabichromevarinic acid (CBCVA) and combinations thereof.

According to some embodiments, said water-soluble alkanol is selected from the group consisting of mono-ols, di-ols, tri-ols and poly-ols.

According to some embodiments, said alkanol comprises at least two (such as two, three, four or more) hydroxyl functional groups and said ester is derived from at least two (such as two, three, four or more) acid forms of a cannabinoid.

According to some embodiments, said alkanol comprises at least two (such as two, three, four or more) hydroxyl functional groups and said ester is derived from at least one (such as one, two, three or more) acid form(s) of a cannabinoid and at least one (such as one, two, three or more) selected from the group consisting of carboxylic acid, fatty acids and mineral acids, such as stearic acid, phosphoric acid, butyric acid or combinations thereof.

According to some embodiments, said alkanol comprises at least two (such as two, three, four or more) hydroxyl functional groups and said ester is derived from at least one (such as one, two, three or more) acid form(s) of cannabinoid and at least one (such as one, two, three or more) arachidonic acid.

As used herein, the term “said ester is derived from” with respect to one or more acid form(s) of a cannabinoid is intended to mean that the ester is a product of a reaction between the cannabinoid and an alkanol.

According to some embodiments, said alkanol is a carbohydrate.

According to some embodiments, said alkanol is a sugar alcohol.

According to some embodiments, said sugar alcohol is selected from the group consisting of arabitol, erythritol, ethylene glycol, fucitol, galactitol, iditol, inositol, isomalt, lactitol, maltitol, maltotetraitol, maltotriitol, mannitol, polyglycitol, ribitol, sorbitol, threitol, volemitol and xylitol, and combinations thereof.

According to some embodiments, said alkanol is glycerol.

According to some embodiments, said at least one acid form of a cannabinoid comprises THCA and/or CBDA; and wherein said water-soluble alkanol is selected from the group consisting of a mono-glyceride, a di-glyceride, a tri-glyceride and combinations thereof.

According to an aspect of some embodiments of the present invention, there is provided a composition as disclosed herein for use in therapy.

According to some embodiments, said therapy comprises treatment of an intestinal disease.

According to some embodiments, the intestinal disease is a functional gastrointestinal disease, such as constipation, irritable bowel syndrome, gas, bloating, gastroesophageal reflux disease or diarrhea.

According to an aspect of some embodiments of the present invention, there is provided a method of treating a subject in need thereof, comprising administering a composition as disclosed herein.

According to some embodiments, said subject is in need of treatment of an intestinal disease.

Examples

| | | | | Alkanol; |
|---|---|---|---|---|
| | | | | Acid 1: |
| | | | | Acid 2: |
| | Acid form of | | | Acid 3 |
| Alkanol | cannabinoid 1 | Acid 2 | Acid 3 | molar ratio |
| Ethylene | CBDA | | | 1:2 |
| glycol | | | | |
| Ethylene | THCA | | | 1:2 |
| glycol | | | | |
| Ethylene | CBGA | | | 1:2 |
| glycol | | | | |
| Ethylene | THCA | CBDA | | 1:1:1 |
| glycol | | | | |
| Ethylene | THCA | Stearic acid | | 1:1:1 |
| glycol | | | | |
| Ethylene | CBDA | Phosphoric | | 1:1:1 |
| glycol | | acid | | |
| Glycerol | CBDA | | | 1:2 |
| Glycerol | THCA | | | 1:3 |
| Glycerol | CBCA | | | 1:2 |
| Glycerol | CBGA | | | 1:2 |
| Glycerol | THCA | | | 1:3 |
| Glycerol | CBDVA | | | 1:2 |
| Glycerol | THCA | CBDA | | 1:1:2 |
| Glycerol | THCA | Stearic acid | | 1:2:1 |
| Glycerol | CBDA | Phosphoric | | 1:1:1 |
| | | acid | | |
| Glycerol | CBDA | Butyric acid | | 1:2:1 |
| Inositol | THCA | | | 1:3 |
| Inositol | THCA | CBDA | | 1:2:2 |
| Inositol | THCA | CBCA | Phosphoric | 1:2:1:1 |
| | | | acid | |
| Inositol | THCA | Phosphoric | | 1:2:3 |
| | | acid | | |
| Sorbitol | CBDA | Phosphoric | | 1:3:1 |
| | | acid | | |
| Sorbitol | THCA | Stearic acid | | 1:2:2 |
| Xylitol | CBCA | | | 1:2 |
| Xylitol | THCA | CBDA | Phosphoric | 1:1:1:1 |
| | | | acid | |

1-15. (canceled)

16. A composition comprising an ester derived from at least one acid form of a cannabinoid and a water-soluble alkanol selected from the group consisting of di-ols, tri-ols and poly-ols.

17. The composition of claim 16, wherein said at least one acid form of a cannabinoid is selected from the group consisting of cannabigerolic acid (CBGA), tetrahydrocannabinolic acid (THCA), cannabidiolic acid (CBDA), cannabichromenic acid (CBCA), cannabigerovarinic acid (CBGVA), tetrahydrocanabivarinic acid (CBCVA), cannabidivarinic acid (CBDVA) and cannabichromevarinic acid (CBCVA) and combinations thereof.

18. The composition of claim 16, wherein said ester is derived from at least two different acid forms of a cannabinoid.

19. The composition of claim 16, wherein said ester is derived from at least one acid form of a cannabinoid and at least one selected from the group consisting of carboxylic acids, fatty acids and mineral acids.

20. The composition of claim 16, wherein said ester is derived from at least one acid form of cannabinoid and at least one arachidonic acid.

21. The composition of claim 16, wherein said alkanol is a carbohydrate.

22. The composition of claim 16, wherein said alkanol is a sugar alcohol.

23. The composition of claim 22, wherein said sugar alcohol is selected from the group consisting of arabitol, erythritol, ethylene glycol, fucitol, galactitol, iditol, inositol, isomalt, lactitol, maltitol, maltotetraitol, maltotriitol, mannitol, polyglycitol, ribitol, sorbitol, threitol, volemitol and xylitol, and combinations thereof.

24. The composition of claim 16, wherein said alkanol is glycerol.

25. The composition of claim 16, wherein said at least one acid form of a cannabinoid comprises THCA and/or CBDA, and wherein said water-soluble alkanol is selected from the group consisting of a mono-glyceride, a di-glyceride, a tri-glyceride and combinations thereof.

26. A method of treating an intestinal disease in a subject in need thereof, comprising administering to the subject the composition of claim 16.
